(19) United States
(12) Patent Application Publication
(10) Pub. No.: US 2025/0248891 A1
(43) Pub. Date: Aug. 7, 2025

(54) Auxiliary standing healthy leisure seat with a motor driving magnetic pole rotation

(71) Applicant: HEYE HEALTH TECHNOLOGY CO., LTD, Huzhou (CN)

(72) Inventors: Shibiao ZHANG, Huzhou (CN); Qunyong YAO, Huzhou (CN); Yanwen FANG, Huzhou (CN); Zhicai FANG, Huzhou (CN)

(73) Assignee: HEYE HEALTH TECHNOLOGY CO., LTD, Huzhou (CN)

(21) Appl. No.: 19/190,874

(22) Filed: Apr. 28, 2025

Related U.S. Application Data
(63) Continuation of application No. PCT/CN2024/081285, filed on Mar. 13, 2024.

(30) Foreign Application Priority Data
Apr. 26, 2023 (CN) 202310457637.2

(51) Int. Cl. A61H 23/02; A61N 2/06

(52) U.S. Cl. CPC A61H 23/0218; A61N 2/06; A61H 2201/0149

(57) ABSTRACT

The present invention discloses a motor-driven rotating magnetic pole assisted standing health and leisure seat, comprising a sofa body and a sofa support connected thereto. The sofa body includes a seat cushion, a backrest, and a footrest. The backrest consists of a lumbar support and headrest from bottom to top, with both the lumbar support and seat cushion equipped with rotating magnetic motors to maximize the high-intensity, high-penetration magnetic field effect. By incorporating rotating magnetic motors in both the seat cushion and backrest, the invention achieves comprehensive coverage of most human body areas. The addition of six magnetic strips expands the magnetic field distribution range and coverage area. Through the combination of dynamic and static magnetic fields, users can better benefit from magnetic therapy, endowing the health and leisure seat with magnetic therapy effect and playing a role in health care.

### RELATED APPLICATIONS

The present patent document is a continuation of PCT Application Serial No. PCT/CN2024/081285, filed Mar. 13, 2024, designating the United States and published in English, which is hereby incorporated by reference.

The present patent document claims the benefit of priority to patent application No. 202310457637.2, filed Apr. 26, 2023, and entitled “An auxiliary standing healthy leisure seat with a motor driving magnetic pole rotation,” the entire contents of each of which are incorporated herein by reference.

### BACKGROUND

### 1. Technical Field

The invention relates to the field of healthy and leisure seating, and in particular to an auxiliary standing healthy and leisure sitting device driven by a motor to rotate magnetic poles.

### 2. Background Information

Massage sofa is one of the health care and physical therapy devices commonly used in people's daily life. According to the settings of the massage devices on the massage sofa, the people sitting on the massage sofa can be massaged on the back, buttocks and other parts, so that the human body can be Relax and relieve fatigue.

At present, diseases such as hypertension, diabetes, and cardiovascular and cerebrovascular diseases have become high-incidence diseases among the Chinese population, and the diseased groups are gradually becoming younger. According to medical surveys, disorders of micro-circulation are the source of all diseases. Traditional Chinese medicine believes that the human body's self-regulatory mechanism is the best treatment method. How to promote human body microcirculation and enhance the human body's self-regulatory mechanism is an inevitable trend in future social development. As one of the forms of health care, magnetic therapy is considered to be a relatively safe human body physical therapy method, but the existing massage sofa does not have the function of magnetic therapy.

### BRIEF SUMMARY

The purpose of the present invention is to solve the problems in the background technology and provide an auxiliary standing health and leisure seat that is driven by a motor to rotate magnetic poles.

The above technical objectives of the present invention are achieved through the following technical solutions:

An auxiliary standing healthy leisure seat that is driven by a motor to rotate magnetic poles. It is characterized in that it includes a sofa body and a sofa support that is cooperatively connected with the sofa body. The sofa body includes a seat cushion, a backrest and a footrest, and the backrest is provided on the seat cushion. At the rear, the footrest is located in front of the seat cushion, and the backrest includes a lumbar support and a headrest from bottom to top. A rotating magnetic motor is installed inside the lumbar support and in the seat cushion. The rotating magnetic motor is 8500˜10000 GS, which effectively exerts high strength and penetration to the best effect. The footrest and the headrest are equipped with magnetic strips.

The present invention adds a rotating magnetic motor to the seat cushion and backrest, which can cover most areas of the human body. By adding 6 magnetic strips, the surface magnetism of the magnetic strips is not less than 2500 Gauss, making the distribution area of the magnetic field wider. The distribution of moving magnets and static magnets allows users to better enjoy the effects of the magnetic field, making the healthy leisure seats have a magnetic therapy effect and play a role in health care.

Preferably, the sofa support includes a lifting device and an electric push rod that drives the lifting device. The electric push rod includes a driving motor, a screw rod, a slider, a shell, a guide rod, a hollow push rod inside, and a first travel switch, a second travel switch, a third travel switch, the drive motor is located in the shell, the screw rod is fixed at the output end of the drive motor. The guide rod is located above the screw rod and parallel to it. The slider is sleeved on the side walls of the screw rod and the guide rod, the slider is threadedly connected to the screw rod and slidingly connected to the guide rod, and the push rod is sleeved on the side wall of the screw rod and the guide rod. The outer side of the screw rod is fixedly connected to the slider, and the first, second and third travel switch arrays are provided on the top of the shell.

The present invention, by rotating the motor, drives the lead screw to rotate, thereby driving the slider to slide on the lead screw, thereby driving the push rod to expand and expand. When the slider touches the first travel switch, the lifting device is driven to work, so that the sofa is in a lying position with a lying Angle of about 170 degrees, so that the human body can sleep easily. When the slider touches the second stroke switch, the body can sleep easily. Drive the lifting device to work, so that the sofa presents a sitting state, when the slider contacts the third stroke switch, drive the lifting device to work, so that the sofa presents an auxiliary standing state, auxiliary standing function, can assist the use of standing up, so that the user's knee is free from the whole weight of the human body, and can easily stand up. It is especially helpful for the elderly and those with knee problems.

Lifting device is a prior art, such as patent CN108125435A, a senior chair mechanical device CN112021847A, a senior chair lifting drive mechanism, seat frame and seat this patent has been documented in detail, so it is not necessary to describe this case. By controlling the electric push rod, the sofa body has the sitting position, lying position and auxiliary standing state.

Preferably, the rotating magnetic motor includes a motor mounting shell with openings at both ends, a brushless motor, a rotating shaft, a magnet, and a magnet mounting shell. One end of the magnet mounting shell is a closed end and the other end is an open end. The magnet mounting shells are provided at both ends of the motor mounting shell and the open end of the magnet mounting shell faces the motor mounting shell. The rotating shaft is located in the motor mounting shell and extends at both ends. The brushless motor is arranged in the motor mounting case, and the two magnets are arranged at both ends of the rotating shaft and are fixedly connected to the outer side wall of the rotating shaft.

The invention uses a brushless motor to drive the magnet to rotate, thereby driving the magnet to rotate, realizing the exchange of positions of N and S-level magnetic fields, and realizing moving magnetism.

By setting magnets on both ends of the brushless motor and a casing on the outside, the entire rotating magnetic motor is then placed on the seat cushion and backrest, so that the magnets can be firmly fixed on the backrest and seat of the leisure chair, and can be massaged as needed. The constant changes in the angle of the chair body can follow the changes in the angle of the chair body during operation.

Preferably, the soft connection of the cloth cover is used to connect to the overall structure to avoid abnormal sounds transmitted by irregular vibrations caused by the rotation of the motor from being felt by the human ear and causing noise pollution.

The product adopts a stepless change method to adjust the different angles of the back and feet, so that the user is always in the most comfortable and desired posture, keeping the entire body and mind in the best state.

The product is designed with superfiber leather, which makes you comfortable to sit on. The superfiber leather has a bright luster, strong moisture absorption and dissipation, and is wear-resistant. It is also refreshing and non-sticky in summer, and will not feel cold in winter.

The rotating magnetic motor is installed under the seat cushion of the chair, and the back is installed at the shoulder, neck and back positions. The fixation method is firm and the operation is smooth. The voltage of the chair adopts 24V safety voltage. The control system is integrated into the control panel of the chair. The operation is simple and clear. The control system Through continuous optimization and improvement, it can be integrated into the control system of the massage chair itself to achieve linkage.

The rotating shaft is provided with support bearings on both sides of the brushless motor. The support bearing is provided on the rotating shaft. The inner ring of the supporting bearing is fixedly connected to the rotating shaft. The supporting bearing is the outer ring is fixedly connected to the side wall of the motor mounting shell, and the distance between the two support bearings is 60˜65 mm.

The present invention lengthens the length of the brushless motor by increasing the distance between the two support bearings, thereby increasing the torque of the brushless motor without changing the overall size of the rotating motor and preventing the magnets at both ends from being attracted by external forces. The magnet is sucked, making the rotating magnetic motor unable to work normally, and at the same time preventing the motor from being pulled and causing damage to the bearings, thereby increasing the life of the rotating motor.

Preferably, both ends of the motor mounting housing are formed with a fixed ring, the outer diameter of the fixed ring is less than the outer diameter of the motor mounting housing port, the fixed ring is provided with a number of conical grooving along the circumferential direction, the cone through the groove is provided with a ball, the cone through the groove is provided with a first retaining ring, the fixed ring is provided with a compression spring. One end of the compression spring is tight to the port of the motor mounting housing, the other end is tight to the first retaining ring, the outer wall of the first retaining ring is fixed connected with a second retaining ring, the open end of the magnet mounting shell is formed with a insertion ring, the outer wall of the insertion ring is provided with a ball card slot for the ball to fall into, the front end of the insertion ring is cone shaped. The port surface of the fixed ring is formed with an elastic sealing ring, and the magnet mounting shell is provided with a sealing card slot matched with the elastic sealing ring.

By sliding the second retaining ring, the compression spring is squeezed, the first retaining ring is displaced and leaves the ball, so that the ball can move, and then the insertion ring is inserted into the fixed ring, the ball is first squeezed out of the inner wall of the fixed ring, the ball is blocked by the second retaining ring and will not fall out of tapered through groove, and then the ball is stuck into the ball clip slot of the insertion ring, and then the hand is loosened. The second retaining ring and the first retaining ring are displaced by the elastic force of the compression spring, so that the first retaining ring will block the outer wall of the cone through the groove, and the elastic sealing ring is stuck into the sealing groove, so as to quickly complete the installation between the motor mounting shell and the magnet mounting shell, and the installation is firm and easy to disassemble and assemble, which is convenient for later maintenance. At the same time, it also strengthens the waterproof performance of the rotary magneto and strengthens the safety performance of the use of healthy leisure seats.

Preferably, the diameter of the tapered through groove increases sequentially from the inner wall to the outer wall of the fixed ring to prevent the balls from falling from the inner wall of the fixed ring.

Preferably, the fixed ring is fixedly connected to a limiting blocking ring at a position close to the tapered through groove, and the first retaining ring can be blocked by the limiting blocking ring.

Preferably, a magnetic isolation piece is provided on a side of the magnet close to the brushless motor, and the magnetic isolation piece is fixedly connected to the side wall of the rotating shaft. The setting of the magnetic isolation sheet prevents the magnetic force of the magnetic body from affecting the work of the rotor and stator structure, and at the same time reduces the dissipation of the magnetic force along the axial direction of the motor, so that the magnetic field gathers in the direction perpendicular to the axial direction of the motor.

Preferably, the seat cushion and lumbar support are also provided with a massage area. The massage area can be a roller or an air bag, which can massage various parts of the human body.

There are two sets of massage air bags distributed overall, and the sitting and back areas produce a massage effect through different frequencies of inflation and deflation.

A total of six flexible magnetic strips on the head and legs are evenly distributed to generate a constant static magnetic field, which is stable and effective.

The chair is controlled by a hand controller and has tilting and standing functions; when tilted down, the angle is about 170 degrees, allowing the human body to sleep easily.

Preferably, the present invention is provided with a one-button reset function, which means that the device can be shut down and reset in any state by pressing the on/off key, and can automatically reset after powering on again.

To sum up, the beneficial effects of the present invention are:
1. The present invention adds a rotating magnetic motor to the seat cushion and backrest, which can cover most areas of the human body. By adding 6 magnetic strips, the distribution area of the magnetic field is wider. Through the distribution of moving magnetism and static magnetism, the magnetic field can be distributed evenly. Users can better enjoy the effects of the magnetic field, making the healthy leisure seats have magnetic therapy effects and play a role in health care.
2. The present invention drives the screw rod to rotate through the rotation of the rotating motor, thereby driving the slider to slide on the screw rod, thereby driving the push rod to expand and contract. When the slider contacts the first travel switch, the lifting device is driven to work, making the sofa appear. Lying position, the lying position angle is about 170 degrees, so that the human body can sleep easily. When the slider contacts the second travel switch, the lifting device is driven to work, making the sofa assume a sitting position. When the slider contacts the third travel switch, drives the lifting device to work, making the sofa appear in an auxiliary standing state. The auxiliary standing function can assist the user in getting up and standing, so that the user's knees are free from the entire weight of the human body and can stand up easily, especially for the elderly and those with knees. There is good help for anyone with questions.
3. In the present invention, the compression spring is squeezed by sliding the second retaining ring, causing the first retaining ring to be displaced and away from the ball, so that the ball can move. Then the insertion ring is inserted into the fixed ring, and the ball is extruded and fixed first. On the inner wall of the ring, the ball is blocked by the second retaining ring and will not fall out of the tapered through groove. Then the ball snaps into the ball slot of the insertion ring. Then the hand is released, so that the second retaining ring and the first retaining ring Due to the elastic force of the compression spring, the displacement occurs, causing the first retaining ring to block the outer wall of the tapered slot, and at the same time, the elastic sealing ring snaps into the sealing slot, thereby quickly completing the connection between the motor mounting shell and the magnet mounting shell. The installation is firm and easy to disassemble and assemble, which facilitates later maintenance. It also strengthens the waterproof performance of the rotating magnetic motor and enhances the safety performance of healthy leisure seating.

### BRIEF DESCRIPTION OF THE DRAWINGS

FIG. 1 is a schematic diagram of the entire invention;

FIG. 2 is a schematic diagram of the state of assisting standing according to the present invention;

FIG. 3 is a schematic diagram of the lying position of the present invention;

FIG. 4 is a schematic diagram of the sitting posture of the present invention;

FIG. 5 is a schematic diagram of the electric push rod of the present invention;

FIG. 6 is a schematic cross-sectional view of the rotating magnetic motor of the present invention;

FIG. 7 is an enlarged schematic diagram of position A in FIG. 6 of the present invention;

FIG. 8 is an enlarged schematic view of B in FIG. 7 of the present invention;

FIG. 9 is a schematic cross-sectional view of the insertion ring of the present invention just inserted into the fixed ring;

FIG. 10 is a schematic diagram of the state of the present invention in which the object assists standing;

FIG. 11 is a schematic diagram of the lying position of the actual object of the present invention;

FIG. 12 is a schematic diagram of the sitting posture of the present invention;

### DETAILED DESCRIPTION OF THE DRAWINGS AND THE PRESENTLY PREFERRED EMBODIMENTS

The following specific examples are only explanations of the present invention and are not intended to limit the present invention. Those skilled in the art can make modifications to the present embodiments without creative contribution as needed after reading this specification. However, as long as the rights of the present invention are all requirements are protected by patent law.

The present invention will be described in detail with examples below in conjunction with the accompanying drawings.

### Example

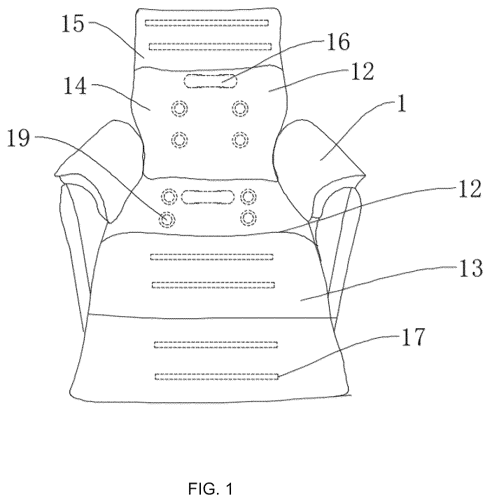

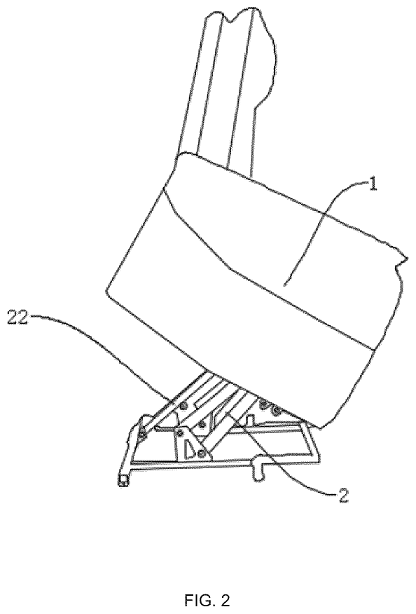

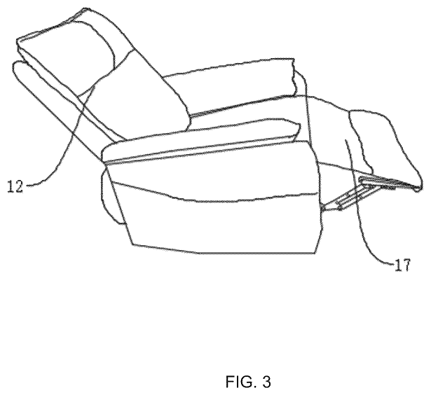

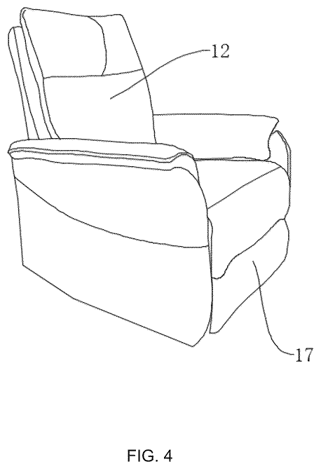

As shown in FIG. 1-5, an auxiliary standing healthy leisure seat is driven by a motor to rotate the magnetic pole. It is characterized in that it includes a sofa body 1 and a sofa support 2 cooperatively connected with the sofa body 1, the sofa body 1 includes a seat cushion 11, a backrest 12 and a footrest 13, the backrest 12 is arranged behind the seat cushion 11, the footrest 13 is arranged in front of the seat cushion 11, the backrest 12 includes a lumbar support 14 and a headrest 15 from bottom to top, rotating magnetic motors 16 are arranged inside both the lumbar support 14 and the seat cushion 11, magnetic strips 17 are arranged inside the footrest 13 and the headrest 15, and massage areas 18 are also provided on the seat cushion 11 and the lumbar support 14.

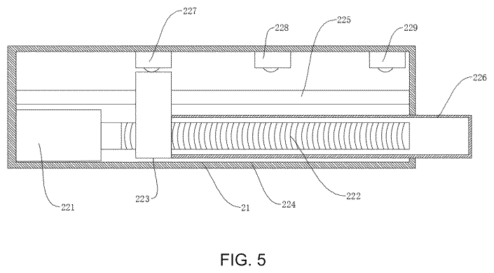

As shown in FIG. 5, the sofa support 2 includes a lifting device 21 and an electric push rod 22 for driving the lifting device, the electric push rod 22 includes a drive motor 221, a screw rod 222, a slider 223, a shell 224, a guide rod 225, a hollow push rod 226, a first travel switch 227, a second travel switch 228, and a third travel switch 229, the drive motor 221 is arranged inside the shell 224, the screw rod 222 is fixed at the output end of the drive motor 221, the guide rod 225 is arranged above the screw rod 222 and is parallel to the screw rod 222, the slider 223 is sleeved on the side walls of the screw rod 222 and the guide rod 225, the slider 223 is in threaded connection with the screw rod 222 and is in sliding connection with the guide rod 225, the hollow push rod 226 is sleeved on the outside of the screw rod 222 and is fixedly connected with the slider 223, and the first travel switch 227, the second travel switch 228, and the third travel switch 229 are arranged in an array on the top of the shell 224.

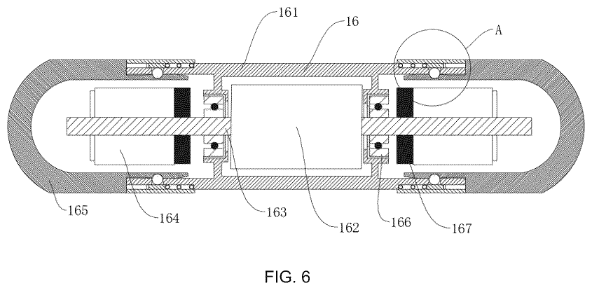

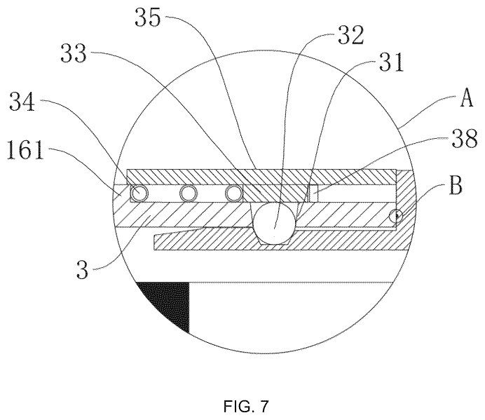

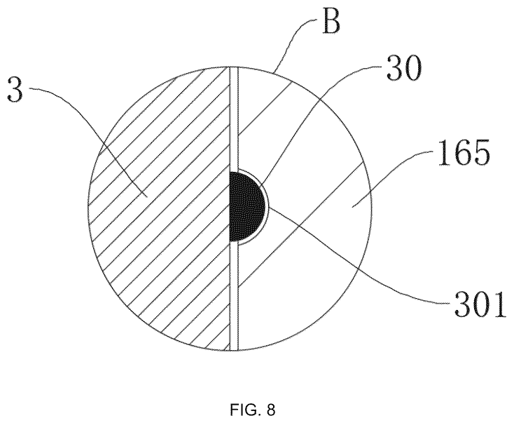

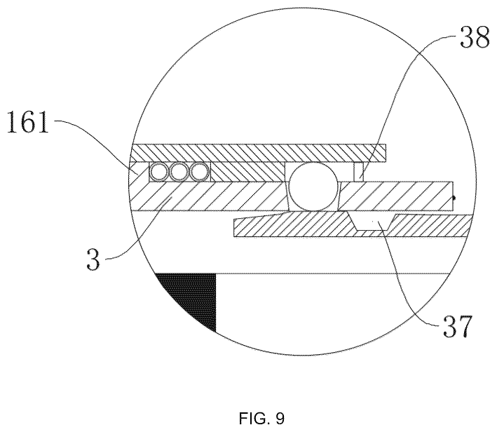

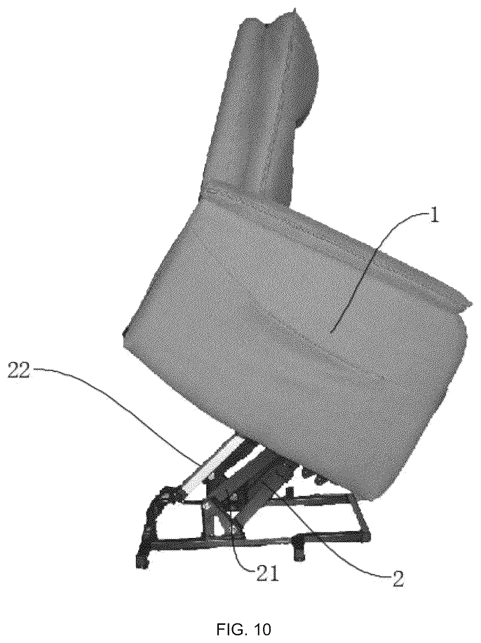

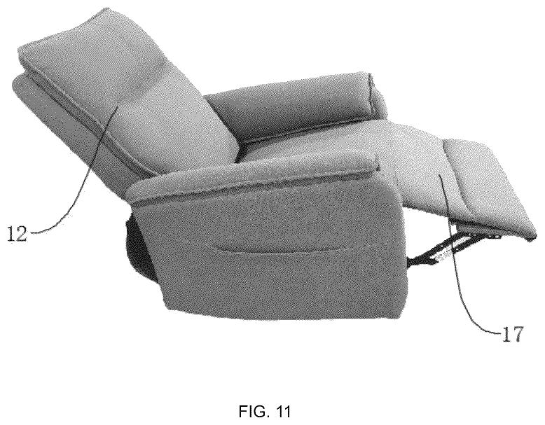

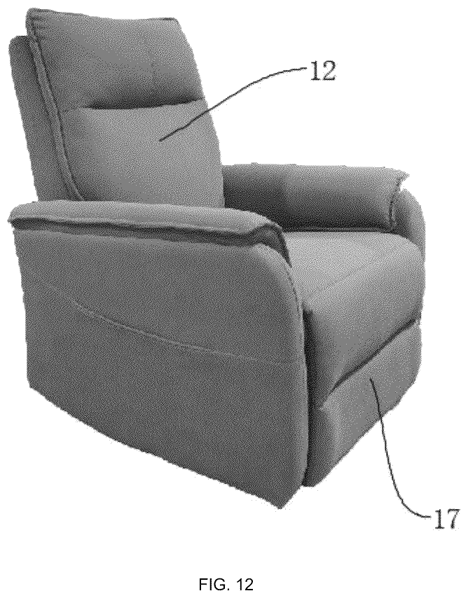

As shown in FIG. 6-9, the rotating magnetic motor 16 includes a motor mounting shell 161 with openings at both ends, a brushless motor 162, a rotating shaft 163, magnets 164, and magnet mounting shells 165, the side of the magnet 164 close to the brushless motor 162 is provided with a magnetic isolation sheet 167, and the magnetic isolation sheet 167 is fixedly connected to the side-wall of the rotating shaft 163; one end of the magnet mounting shell 165 is a closed end and the other end is an open end, two magnet mounting shells 165 are arranged at both ends of the motor mounting shell 161 with the open ends of the magnet mounting shells 165 facing the motor mounting shell 161, the rotating shaft 163 is arranged inside the motor mounting shell 161 with both ends extending into the magnet mounting shells 165, the brushless motor 162 is arranged inside the motor mounting shell 161, and two magnets 164 are arranged at both ends of the rotating shaft 163 and fixedly connected to the outer side-wall of the rotating shaft 163, the rotating shaft 163 is provided with support bearings 166 on both sides of the brushless motor 162, the support bearings 166 are arranged on the rotating shaft 163, the inner rings of the support bearings 166 are fixedly connected with the rotating shaft 163, the outer rings of the support bearings 166 are fixedly connected with the side-wall of the motor mounting shell 161, and the distance between the two support bearings 166 is 60-65 mm, the two ends of the motor mounting shell 161 are each formed with a fixed ring 3, the outer diameter of the fixed ring 3 is smaller than that of the port of the motor mounting shell 161, the fixed ring 3 is provided with several tapered through grooves 31 along the circumferential direction, there are balls 32 in the tapered through grooves 31, a first retaining ring 33 is arranged outside the tapered through grooves 31, a compression spring 34 is sleeved on the fixed ring 3, one end of the compression spring 34 abuts against the port of the motor mounting shell 161 and the other end abuts against the first retaining ring 33, the outer side-wall of the first retaining ring 33 is fixedly connected with a second retaining ring 35, the open end of the magnet mounting shell 165 is formed with an insertion ring 36, the outer side-wall of the insertion ring 36 is provided with a ball slot 37 for the balls to fall into, and the front end of the insertion ring 36 is cone-shaped, the port surface of the fixed ring 3 is formed with an elastic sealing ring 30, and the magnet mounting shell 165 is provided with a sealing groove 301 that cooperates with the elastic sealing ring, the diameter of the tapered through groove 31 increases sequentially from the inner wall to the outer wall of the fixing ring 3, the fixed ring 3 is fixedly connected to a limited stop ring 38 at a position close to the tapered through groove 31.
Motor input voltage: DC12V Current: 10˜50 mA Speed: 600 rpm,
Power: 0.7 W,
Rotating magnetic field: 8500˜10000 GS Noise: below 30 dB,
Motor outer diameter: 56 mm Motor length: 140 mm.
Working principle: As shown in FIG. 1-12, The rotation of the rotating motor 221 drives the screw rod 222 to rotate, which in turn causes the slider 223 to slide on the screw rod 222, driving the hollow push rod 226 to extend and retract. When the slider 223 touches the first travel switch 227, it activates the lifting device, making the sofa assume a lying-down position with a lying-down angle of about 170 degrees, allowing the human body to sleep comfortably. When the slider 223 touches the second travel switch 228, it activates the lifting device, causing the sofa to take on a sitting position. When the slider 223 touches the third travel switch 229, it activates the lifting device 21, enabling the sofa to enter an auxiliary standing state. The auxiliary standing function helps the user to stand up, relieving the knees of the user from bearing the entire body weight and allowing for easy standing, which is particularly beneficial for the elderly and those with knee problems. When dynamic magnetic therapy is required, the brushless motor 162 in the rotating magnetic motor 16 rotates, driving the magnet 164 to rotate and achieving the exchange of the N and S class magnetic fields. By adding six magnetic strips with a surface magnetic field of no less than 2500 Gauss, the distribution area and scope of the magnetic field are expanded. Through the distribution of dynamic and static magnetic fields, the user can better experience the effects of the magnetic field, endowing the health and leisure seat with magnetic therapy effects and serving a health-care function. The auxiliary standing-type health and leisure seat in this case not only has a massage function but also can perform magnetic therapy and assist the user in standing up, enhancing its applicability.

1. An auxiliary standing healthy leisure seat with a motor driving magnetic pole rotation, characterized in that:
it includes a sofa body (1) and a sofa support (2) cooperatively connected with the sofa body (1), the sofa body (1) includes a seat cushion (11), a backrest (12) and a footrest (13), the backrest (12) is arranged behind the seat cushion (11), the footrest (13) is arranged in front of the seat cushion (11), the backrest (12) includes a lumbar support (14) and a headrest (15) from bottom to top, rotating magnetic motors (16) are arranged inside both the lumbar support (14) and the seat cushion (11), and magnetic strips (17) are arranged inside the footrest (13) and the headrest (15).

2. An auxiliary standing healthy leisure seat with a motor driving magnetic pole rotation according to claim 1, characterized in that:
the sofa support (2) includes a lifting device (21) and an electric push rod (22) for driving the lifting device, the electric push rod (22) includes a drive motor (221), a screw rod (222), a slider (223), a shell (224), a guide rod (225), a hollow push rod (226), a first travel switch (227), a second travel switch (228), and a third travel switch (229), the drive motor (221) is arranged inside the shell (224), the screw rod (222) is fixed at the output end of the drive motor (221), the guide rod (225) is arranged above the screw rod (222) and is parallel to the screw rod (222), the slider (223) is sleeved on the side walls of the screw rod (222) and the guide rod (225), the slider (223) is in threaded connection with the screw rod (222) and is in sliding connection with the guide rod (225), the hollow push rod (226) is sleeved on the outside of the screw rod (222) and is fixedly connected with the slider (223), and the first travel switch (227), the second travel switch (228), and the third travel switch (229) are arranged in an array on the top of the shell (224).

3. An auxiliary standing healthy leisure seat with a motor driving magnetic pole rotation according to claim 1, characterized in that:
the rotating magnetic motor (16) includes a motor mounting shell (161) with openings at both ends, a brushless motor (162), a rotating shaft (163), magnets (164), and magnet mounting shells (165), one end of the magnet mounting shell (165) is a closed end and the other end is an open end, two magnet mounting shells (165) are arranged at both ends of the motor mounting shell (161) with the open ends of the magnet mounting shells (165) facing the motor mounting shell (161), the rotating shaft (163) is arranged inside the motor mounting shell (161) with both ends extending into the magnet mounting shells (165), the brushless motor (162) is arranged inside the motor mounting shell (161), and two magnets (164) are arranged at both ends of the rotating shaft (163) and fixedly connected to the outer side-wall of the rotating shaft (163).

4. An auxiliary standing healthy leisure seat with a motor driving magnetic pole rotation according to claim 3, characterized in that:
the rotating shaft (163) is provided with support bearings (166) on both sides of the brushless motor (162), the support bearings (166) are arranged on the rotating shaft (163), the inner rings of the support bearings (166) are fixedly connected with the rotating shaft (163), the outer rings of the support bearings (166) are fixedly connected with the side-wall of the motor mounting shell (161), and the distance between the two support bearings (166) is 60-65 mm.

5. An auxiliary standing healthy leisure seat with a motor driving magnetic pole rotation according to claim 4, characterized in that:
the two ends of the motor mounting shell (161) are each formed with a fixed ring (3), the outer diameter of the fixed ring (3) is smaller than that of the port of the motor mounting shell (161), the fixed ring (3) is provided with several tapered through grooves (31) along the circumferential direction, there are balls (32) in the tapered through grooves (31), a first retaining ring (33) is arranged outside the tapered through grooves (31), a compression spring (34) is sleeved on the fixed ring (3), one end of the compression spring (34) abuts against the port of the motor mounting shell (161) and the other end abuts against the first retaining ring (33), the outer side-wall of the first retaining ring (33) is fixedly connected with a second retaining ring (35), the open end of the magnet mounting shell (165) is formed with an insertion ring (36), the outer side-wall of the insertion ring (36) is provided with a ball slot (37) for the balls to fall into, and the front end of the insertion ring (36) is cone-shaped.

6. An auxiliary standing healthy leisure seat with a motor driving magnetic pole rotation according to claim 5, characterized in that:
the port surface of the fixed ring (3) is formed with an elastic sealing ring (30), and the magnet mounting shell (165) is provided with a sealing groove (301) that cooperates with the elastic sealing ring.

7. An auxiliary standing healthy leisure seat with a motor driving magnetic pole rotation according to claim 5, characterized in that:
the diameter of the tapered through groove (31) increases sequentially from the inner wall to the outer wall of the fixing ring (3).

8. An auxiliary standing healthy leisure seat with a motor driving magnetic pole rotation according to claim 5, characterized in that:
the fixed ring (3) is fixedly connected to a limited stop ring (38) at a position close to the tapered through groove (31).

9. An auxiliary standing healthy leisure seat with a motor driving magnetic pole rotation according to claim 3, characterized in that:
the side of the magnet (164) close to the brushless motor (162) is provided with a magnetic isolation sheet (167), and the magnetic isolation sheet (167) is fixedly connected to the side-wall of the rotating shaft (163).

10. An auxiliary standing healthy leisure seat with a motor driving magnetic pole rotation according to claim 1, characterized in that:
the seat cushion (11) and lumbar support (141) are also provided with a massage area (18).
